SEQUENCE LISTING (12 sequences)

SEQ ID NO: 1 - DNA, 24 bp, Artificial Sequence
  Description of Artificial Sequence Primer
atgaattaccaagtcaatggttac

SEQ ID NO: 2 - DNA, 22 bp, Artificial Sequence
  Description of Artificial Sequence Primer
cataaccagtcggtacagctac

SEQ ID NO: 3 - DNA, 20 bp, Artificial Sequence
  Description of Artificial Sequence Primer
gaagctattcgtcacgttcg

SEQ ID NO: 4 - DNA, 22 bp, Artificial Sequence
  Description of Artificial Sequence Primer
ctgtagaaaatcctagctggag

SEQ ID NO: 5 - DNA, 24 bp, Artificial Sequence
  Description of Artificial Sequence Primer
ggccgcaaattgcacaatttgctc

SEQ ID NO: 6 - DNA, 24 bp, Artificial Sequence
  Description of Artificial Sequence Primer
ccatgtcagccgcaggaagaagag

SEQ ID NO: 7 - DNA, 24 bp, Artificial Sequence
  Description of Artificial Sequence Primer
tgcctctgcattctttggaatgtc

SEQ ID NO: 8 - DNA, 24 bp, Artificial Sequence
  Description of Artificial Sequence Primer
tatgcgtcaatgtgcttgttcagc

SEQ ID NO: 9 - DNA, 24 bp, Artificial Sequence
  Description of Artificial Sequence Primer
catggcaaggaggaacttagattc

SEQ ID NO: 10 - DNA, 24 bp, Artificial Sequence
  Description of Artificial Sequence Primer
cacggtggcagcattgttattagg

SEQ ID NO: 11 - DNA, 24 bp, Artificial Sequence
  Description of Artificial Sequence Primer
acaccaatagtggtccagatgacc

SEQ ID NO: 12 - DNA, 24 bp, Artificial Sequence
  Description of Artificial Sequence Primer
cgccgtagggaagtgaagcttctg

(19) United States
(12) Patent Application Publication
(10) Pub. No.: US 2005/0233314 A1
(43) Pub. Date: Oct. 20, 2005

(54) Sensitive and quantitative detection of pathogens by real-time nested PCR

(75) Inventors: Jyh-Lyh Juang, Taipei City (TW); Shih Sheng Jiang, Ta-Li City (TW)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: National Health Research Institutes

(21) Appl. No.: 10/879,734

(22) Filed: Jun. 30, 2004

Related U.S. Application Data
(60) Provisional application No. 60/483,179, filed on Jun. 30, 2003.
(60) Provisional application No. 60/484,101, filed on Jun. 30, 2003.

(51) Int. Cl. C12Q 1/70; C12Q 1/68; C12P 19/34

(52) U.S. Cl. 435/5; 435/6; 435/91.2

(57) ABSTRACT

The invention describes compositions and methods for detecting and/or quantifying an RNA or DNA pathogen in a sample. The method comprises a two-round real-time nested PCR, which allows detection of less than 10 copies of RNA or DNA of the pathogen in a sample. The method of the invention is useful for fast, reliable, and sensitive detection and/or quantification of SARS-CoV in a sample.

### RELATED APPLICATION

This application claims the benefit of U.S. Provisional Application No. 60/483,179, filed Jun. 30, 2003, the contents of which are relied on are incorporated herein by reference. This application also relates to U.S. Application Ser. No. ______, filed concurrently herewith, which claims the benefit of U.S. Provisional Application Ser. No. 60/484,101, entitled “Apparatus and Methods for Computer Aided Primer Design and Compositions for Detection of the SARS Coronavirus,” to Chao Agnes Hsiung, Chung-Yen Lin, Chen-Zen Lo, Chi-Shiang Cho, and Jyh-Yuan Yang, filed Jun. 30, 2003, both of which are incorporated entirely by reference.

### FIELD OF THE INVENTION

The present invention relates to methods for detecting and/or quantifying RNA or DNA pathogens in a sample. The method can be used to detect and/or quantify the etiological agent of severe acute respiratory syndrome (SARS). The method employs real-time nested polymerase chain reaction (PCR). The present invention further relates to compositions and kits for detecting and/or quantifying RNA or DNA pathogens in a sample.

### BACKGROUND

PCR, the polymerase chain reaction, is one of the most popular methods in biological and biomedical benchwork today. The advantage of PCR is that it amplifies small amounts of nucleic acids by millions to billions. It opened a new age in genetic analysis on a molecular level (Glennon, M. and Cormican, M. (2001) Expert. Rev. Mol. Diagn., 1:163-174; Jain, K. K. (2002) Med. Device Technol., 13:14-18). Indeed PCR finds application as a research and diagnostic tool, for example, in detecting the presence of pathogenic virus or bacteria.

Although PCR is time saving and relatively sensitive, false negative signals often arise in samples with very low copy number of a pathogen. This becomes problematic when an outbreak of a disease or infection needs to be detected at the very early stages of infection, or during a latent period where pathogenic levels are extremely low, in order to prevent and contain the spread of the infection or disease. One such recent example is the severe acute respiratory syndrome (SARS).

In late 2002, cases of life-threatening respiratory disease with no identifiable cause were reported in China, Vietnam, Canada, and Hong Kong. Patients exhibited fever, dry cough, dyspnea, headache, and hypoxemia and death resulted from progressive respiratory failure due to alveolar damage (Tsang K W et al., (2003) N. Engl. J. Med. 348:1975-1983). The syndrome was designated severe acute respiratory syndrome (SARS) and has become a serious global endemic infectious disease in over 30 countries (Drosten C S et al., (2003) N. Engl. J. Med. 348:1967-1976; Ksiazek T G et al., (2003) N. Engl. J. Med. 348:1953-1966; Peiris J S et al., (2003) Lancet 361:1767-1772). Indeed, approximately four percent of patients with SARS have died worldwide (see http://www.who.int/csr/sarscountry/2003 04 04/en/).

The etiological agent of SARS is a novel coronavirus and was termed SARS-associated coronavirus (SARS-CoV). The complete genome sequence of the new coronavirus has recently been determined (Marra M A et al., (2003) Science 300:1399-1404; Rota P A et al., (2003) Science 300:1394-1399). The genome sequence data available so far from several SARS-CoV strains reveal that the novel agent does not belong to any of the known groups of coronaviruses (Drosten C S et al., (2003) N. Engl. J. Med. 348:1967-1976; Ksiazek T G et al., (2003) N. Engl. J. Med. 348:1953-1966; Marra M A et al., (2003) Science 300:1399-1404; Rota P A et al., (2003) Science 300:1394-1399). SARS-CoV is only moderately related to the human coronaviruses and it has been proposed that SARS-CoV is an animal virus that has recently developed the ability to productively infect humans (Ludwig B et al., (2003) Intervirology 46:71-78).

The coronaviruses are members of a family of large, enveloped, positive-stranded RNA viruses that replicate in the cytoplasm of animal host cells (Sidell S et al., (1983) J. Gen. Virol. 64:761-776). Until the discovery of SARS-CoV, there were three groups of coronaviruses. Groups 1 and 2 contain mammalian viruses and group 3 contains only avian viruses. The coronaviruses are associated with a variety of diseases in humans and domestic animals but only the animal coronaviruses are known to cause severe disease. The human strains have only previously been associated with mild respiratory illnesses. SARS-CoV appears to define a fourth group of coronavirus and is the first coronavirus that regularly causes severe disease in humans.

In the absence of a vaccine or effective therapeutic drugs, the key to preventing and controlling further epidemics is to isolate the suspected cases and to implement strict quarantine policies. Unfortunately, isolation and quarantine of individuals and communities exposed to SARS have failed to contain the spread of the disease because of the lack of methods for detecting SARS at an early stage, highlighting the demand for a sensitive early diagnostic method for detection of the virus.

Single-round real-time reverse transcription (RT)-PCR detection has been used for the detection of pathogens (Drosten C S et al., (2003) N. Engl. J. Med. 348:1967-1976; Poon L L et al., (2003) Clin. Chem. 49:953-955). “Real-time” detection allows one to measure the accumulation of PCR product during the course of the reaction, rather than simply analyzing the final product amount following the course of sequential cycles of amplification. However, these methods are generally very inconsistent in the clinical diagnostic setting when different primer sets or different detection methods are utilized. In particular, because the concentration of extracted viral RNA from early infection samples is often very low, the aforementioned problems usually become worse. Indeed, the single-round real-time RT-PCR method which was suggested by the World Health Organization (WHO) for the detection of SARS-CoV (http://www.who.int/csr/sars/diagnostictests/en/) is unable to detect the virus when present in less than 10 copies. False-negatives due to lack of sensitivity of the assay may mislead the clinician to discharge an early-infected individual from the hospital.

Conventional (non real-time) two-round PCR using nested primers in the second round has also been suggested to enhance both the specificity and sensitivity of the assay (Berg J et al., (2001) J. Clin. Virol. 20:71-75; Bialek R et al., (2002) Clin. Diagn. Lab. Immunol. 9:461-469; Ratge D et al., (2002) J. Clin. Virol. 24:161-172; Koenig M et al. (2003) Diagn. Microbiol. Infect. Dis. 46:35-37; Zeaiter Z et al., (2003) J. Clin. Microbiol. 41:919-925). However, the conventional two-round PCR has not been favored as a quantitative assay. In order to accurately determine the amount of initial substrate, the amount of PCR product produced must be measured before the formation of reaction products plateaus. Because the products typically are not analyzed until the completion of the PCR assay, these conventional PCR assays would require lengthy processing times and “trials and errors.” Moreover, they would require labor-intensive handling procedures in a Biosafety Level (BSL) 2 facility and therefore are incompatible for large scale screening of samples.

Therefore, there is a need for an improved diagnostic method to detect SARS-CoV and other pathogens in patients.

### SUMMARY OF THE INVENTION

The present invention provides a method for detecting RNA or DNA pathogens in a sample. The present invention also provides a method for quantifying RNA or DNA pathogens in a sample. Both methods comprise subjecting a sample suspected of containing an RNA or DNA pathogen, to real-time nested PCR. “Real-time” detection allows one to measure the accumulation of amplified product during the course of the reaction, rather than simply analyzing the final product amount following the course of sequential cycles of amplification. “Nested” PCR generally comprises a two-staged polymerase chain reaction process. In a first-stage polymerase chain reaction, a pair of “outer” oligonucleotide primers are used to amplify a first nucleotide sequence. In a second-stage polymerase chain reaction, a second set of “inner” or “nested” oligonucleotide primers are used to amplify a smaller second nucleotide sequence that is contained within the first nucleotide sequence. In the methods of the invention, both stages of nested PCR are based on real-time amplification. The method of the invention is capable of detecting or quantifying less than 10 copies of RNA or DNA in a sample. The method of the invention may be used to detect or quantify SARS-CoV in a sample.

The present invention also provides a composition comprising a first amplified product obtained from subjecting a sample containing an RNA pathogen to real-time reverse transcriptase-polymerase chain reaction (RT-PCR) using at least two “outer” oligonucleotide primers, at least two “nested” oligonucleotide primers, and a compound that detects a second amplified product obtained from nested PCR amplification of the first amplified product. The present invention also provides a composition comprising a first amplified product obtained from subjecting a sample containing a DNA pathogen to real-time polymerase chain reaction (PCR) using at least two “outer” oligonucleotide primers, at least two “nested” oligonucleotide primers, and a compound that detects a second amplified product obtained from nested PCR amplification of the first amplified product. The compound that detects a second amplified product may be a fluorogenic molecule that detects double-stranded DNA.

The present invention further provides a kit for detecting or quantifying a RNA or DNA pathogen in a sample by real-time nested PCR comprising at least two “outer” oligonucleotide primers complementary to the nucleotide sequence of the pathogen and used to obtain a first amplified product, at least two “nested” oligonucleotide primers used to obtain a second amplified product, and a first compound that detects the first amplified product and a second compound that detects the second amplified product. The first and second compounds may be the same or different and may be a fluorogenic molecule that detects double-stranded DNA.

### BRIEF DESCRIPTION OF THE DRAWINGS

FIGS. 1A-1C are graphs showing the sensitivities of real-time first round PCR and real-time nested PCR. FIG. 1A shows the results of real-time first-round RT-PCR carried out in the LightCycler RNA master SYBR green 1 reaction mixture. Viral RNAs were serially diluted to 10³ to 10⁰ from stock viral RNA. The negative control was 2 μl of deionized water instead of viral RNA. FIG. 1B shows the results of real-time nested PCR carried out using LightCycler FastStart DNA master SYBR green 1 reagent kit. 2 μl of amplicon from each first-round real-time RT-PCR (indicated by the starting RNA copy) was recovered and added into each nested amplification reaction. FIG. 1C shows the melting analysis of real-time nested PCR products. The melting phenomena as shown occurred consistently over a 4-log range (1 to 1000 copies) of nested PCR amplicons but was not detected in the negative control (-⋄-). The insert shows that melting peaked at around 84.1±0.4° C.

FIG. 2 is a 2% agarose gel electrophoresis analysis confirming the size of amplicons from real-time nested PCR. RNA copy numbers in each sample are indicated above each lane. NC, negative control; L: molecular marker.

FIG. 3 is a graph showing the linear amplification of real-time nested PCR. The starting viral RNA concentration was plotted against second round cycle number. R, regression coefficient; error, square error.

### DESCRIPTION OF THE EMBODIMENTS

The present invention provides a novel real-time nested PCR method for detecting or quantifying an RNA or DNA pathogen, in particular, SARS-CoV. The real-time nested PCR method of the invention comprises two real-time PCR stages. “Real-time amplification,” “real-time PCR,” “real-time RT-PCR,” or “real-time nested PCR” all refer to amplification techniques, particularly PCR, wherein the amount of amplification product formed can be monitored during the amplification process. A nonexhaustive list of real-time PCR techniques include those described in Heid C A et al., (1996) Genome Research 6:986-994; Gibson UEM et al., (1996) Genome Research 6:995-1001; Holland P M et al., (1991) PNAS 88:7276-7280; Livak K J et al., (1995) PCR Methods and Applications at 357-362; U.S. Pat. No. 5,210,015 (Gelfand); U.S. Pat. No. 5,538,848 (Livak, et al.); and U.S. Pat. No. 5,863,736 (Haaland). Kits for real-time PCR analysis are also commercially available, such as the LlghtCycler RNA Master SYBR Green I kit (Roche Diagnostics GmbH, Germany).

If a sample is suspected of containing an RNA pathogen, such as SARS-CoV, the first amplification step in the method of the invention comprises a real-time reverse transcriptase-PCR (RT-PCR) reaction. If a sample is suspected of containing a DNA pathogen, the DNA pathogen may be directly amplified by real-time PCR. RT-PCR is well known in the art. Reverse transcription is used to prepare template DNA from an initial RNA sample, e.g. mRNA. Template DNA is then amplified using PCR to produce a sufficient amount of amplified product. The RT and PCR steps of DNA amplification may be carried out as a two step or one step process.

In a two step process, the first step involves synthesis of first strand cDNA with a reverse transcriptase, e.g. MMLV-RT, followed by a second PCR step. In certain protocols, these steps are carried out in separate reaction tubes. In these two tube protocols, an aliquot of the reverse transcription product is placed into a second PCR tube and subjected to PCR amplification.

In another two-step process, both RT and PCR are carried out in the same tube using a compatible RT and PCR buffer. In certain embodiments of single tube protocols, reverse transcription is carried out first, followed by addition of PCR reagents to the reaction tube and subsequent PCR.

In an effort to further expedite and simplify RT-PCR procedures, a variety of one step RT-PCR protocols have been developed. See e.g. Blain et al., (1993) J. Biol. Chem. 5:23585-23592; Blain et al., (1995) J. Virol. 69:4440-4452; Sellner et al., (1994) J. Virol. Method. 49:47-58; PCR, ESSENTIAL TECHNIQUES (ed. J. F. Burke, J. Wiley & Sons, New York) (1996) pp. 61-63 and 80-81. Real-time RT-PCR may be performed by any of the methods described in the references cited above.

The term “primer” refers to a single stranded oligonucleotide sequence complementary to the nucleic acid strand to be copied and capable of acting as a point of initiation for synthesis of a primer extension product. The oligonucleotide primers used in the first stage, or first round, PCR reaction comprise at least a pair of “outer” oligonucleotide primers. The pair comprises two primers that flank a specific “target” nucleotide sequence and that hybridize to opposite strands of the specific target nucleotide sequence such that upon repeated elongation of the primers, that specific nucleotide sequence is amplified to produce a “first amplified product.” The “nested” oligonucleotide primers in the second stage, or second round, PCR reaction also comprise of two primers that hybridize to opposite strands of a target nucleotide sequence. The nested primers, however, flank a nucleotide sequence found within the first amplified product produced in the first round PCR reaction. Thus, the second round PCR reaction produces a smaller “second amplified product” from the first amplified product produced in the first round PCR reaction.

The length and the sequence of the oligonucleotide primers must be such that they prime the synthesis of the extension products. In an embodiment of the invention, the primer is from about 5 to about 50 nucleotides long. Specific length and sequence will depend on the complexity of the required DNA or RNA targets, as well as on conditions such as temperature and ionic strength. Methods for the synthesis of these primers are available in the art. See e.g., Sambrook et al. (1989) Molecular Cloning: A Laboratory Manual (2d ed.; Cold Spring Harbor Laboratory: Plainview, N.Y.), herein incorporated by reference. Other methods for selecting primers are taught in U.S. Provisional Application No. 60/484,101, filed Jun. 30, 2003, and subsequently filed concurrently herewith as U.S. Nonprovisional Application Ser. No. ______, both of which are herein incorporated by reference in their entirety. The oligonucleotide primers need not exhibit an exact match with the corresponding template sequence as discussed in Kwok S et al., (1990) Nucleic Acids Research 18:999-1005. The oligonucleotide primers may also comprise nucleotide analogues such as phosphorothiates (Matsukura et al., (1987) Proc. Natl. Acad. Sci. USA 84:7706-10), alkylphosphorothiates (Miller et al., (1979) Biochemistry 18:5134-43) or peptide nucleic acids (Nielsen et al., (1991) Science 254:1497-1500; Nielsen et al., (1993) Nucleic Acids Res. 21:197-200) or may contain intercalating agents (Asseline et al., (1984) Proc. Natl. Acad. Sci. USA 81:3297-301).

In the first round real-time PCR reaction, the target nucleotide sequence is the RNA or DNA sequence of a pathogen. RNA pathogens include, but are not limited to RNA viruses, and DNA pathogens include, but are not limited to DNA viruses. Examples of RNA viruses include the Togavirus family of RNA viruses, which includes the genus alphavirus, which in turn, includes many important viral species such as Sindbis virus, Semliki Forest virus, and pathogenic members such as the Venezuelan, Eastern and Western equine encephalitis virus. Another pathogenic Togavirus is the rubella virus, a virus closely related to the alphaviruses and the causative agent for German measles. Coronaviruses (which includes SARS-CoV) and astroviruses (associated with pediatric diarrhea) are also pathogenic RNA viruses. The Picornaviruses are also RNA viruses which include the Poliovirus, Coxsackievirus, Echovirus, Enterovirus and Rhinovirus. DNA viruses include Paroviruses, Papovaviruses which include the Papilloma viruses which can infect rabbits and the Polyomaviruses which infect primates, Adenoviruses, Herpes viruses, and hepadnaviruses. Others are known in the art. The sequences of all known and sequenced viruses are publicly available at www.ncbi.nih.gov/genomes/VIRUSES/10239.html.

Other DNA pathogens include microbes such as bacteria and yeast. Exemplary microbes include the Bacillus, Chlamydia, and Streptococcus species. The genome sequences of microbes are publicly available at www.ncbi.nlm.nih.gov/genomes/MICROBES/complete.html. Retroviruses may also be detected by the method of the invention. A retrovirus is an RNA virus that has a DNA intermediate step during replication. Retroviruses include the human immunodeficiency virus (HIV). Others are known in the art and sequences of various retrovirus genomes can be found at www.ncbi.nlm.nih.gov/retroviruses/.

In an embodiment of the invention, the target nucleotide sequence is the Spike (S) (also identifiable as the E2 gene, gi|29826277:21477-25244); Matrix (M) (also identifiable as gi|29826277:26383-27048); nucleocapsid (N); and orf1ab polyprotein (P) (also identifiable as gi|29836505) gene of SARS-CoV. As an example of the real-time nested PCR of the invention, the outer and nested PCR primers may be designed to amplify a 195 base pair fragment and a 110 base pair fragment, respectively.

In order to further reduce false positives by cross-priming to contaminating non-target sequences, candidate primers may be checked by BLAST searches against public databases of sequences. Primers may be selected for uniqueness when compared to known sequences, thereby minimizing the likelihood of false-positives.

The first round real-time PCR reaction may be used to follow the course of the PCR reaction to ensure linear amplification of the target sequence. Thus, the second round real-time nested PCR reaction using the first amplified product as the target sequence allows detection and/or quantification of the amount of pathogen that was present in the initial sample.

Amplified products produced by real-time nested PCR may be detected by any of the methods known in the art. In an embodiment of the invention, the amplified products are detected by fluorescence of a compound such as SYBR Green (Roche), which binds to double-stranded DNA. Use of such fluorescent compounds allows the monitoring of the reaction so that conditions may be optimized to control the amplification process. In another embodiment of the invention, gold nanoparticles derivatized with thiol-modified oligonucleotide primers may be designed to bind complementary nucleotide targets as described in Storhoff J J et al., (2004) Biosens. Bioelectron. 19:875-883. Amplification of the gold nanoparticle primer with silver allows for detection and quantitation by measuring evanescent wave-induced light scatter. Other methods of detection are described in Heid C A et al., (1996) Genome Research 6:986-994; Gibson UEM et al., (1996) Genome Research 6:995-1001; Holland P M et al., (1991) PNAS 88:7276-7280; Livak K J et al., (1995) PCR Methods and Applications at 357-362; U.S. Pat. No. 5,210,015 (Gelfand); U.S. Pat. No. 5,538,848 (Livak, et al.); and U.S. Pat. No. 5,863,736 (Haaland).

The initial concentration of the RNA or DNA pathogen in a sample may be determined by establishing a standard curve that correlates input RNA or DNA amount to the amount of amplified product detected after completion of the real-time nested PCR method of the invention. The amount of amplified product detected in a test sample, such as a sample suspected of containing SARS-CoV, may be compared to the standard curve and the amount of initial RNA or DNA contained in the sample may be determined.

The present invention provides a composition comprising at least two oligonucleotide primers complementary to the nucleotide sequence of a pathogen to be amplified and/or at least two nested oligonucleotide primers complementary to the nucleotide sequence amplified, for detecting or quantifying DNA or RNA sequences of pathogens. The present invention also provides a composition comprising: (a) a first amplified product obtained from subjecting a sample containing an RNA pathogen to real-time RT-PCR using at least two oligonucleotide primers complementary to a nucleotide sequence of the RNA pathogen; (b) at least two nested oligonucleotide primers complementary to the nucleotide sequence of the first amplified product; and (c) a compound that detects a second amplification product obtained from nested PCR amplification of the first amplified product using the nested oligonucleotide primers of (b). The present invention further provides a composition comprising: (a) a first amplified product obtained from subjecting a sample containing an DNA pathogen to real-time polymerase chain reaction (PCR) using at least two oligonucleotide primers complementary to a nucleotide sequence of the DNA pathogen; (b) at least two nested oligonucleotide primers complementary to the nucleotide sequence of the first amplified product; and (c) a compound that detects a second amplification product obtained from nested PCR amplification of the first amplified product using the nested oligonucleotide primers of (b). As set forth above, RNA pathogens include togavirus, coronavirus, astrovirus, picornavirus, and retrovirus. In an embodiment, the RNA pathogen is SARS-CoV or human immunodeficiency virus. Also as set forth above, DNA pathogens include DNA viruses such as parovirus, papovavirus, polyomavirus, adenovirus, herpes virus, hepadnavirus, and microbes such as Bacillus, Chlamydia, and Streptococcus. A compound that detects a second amplification product includes a fluorogenic molecule that binds to double-stranded DNA, such as SYBR Green (Roche), or a silver molecule that emits evanescent wave induced light scatter. The first amplified product may be a product of linear amplification of the nucleotide sequence of the pathogen present in the sample.

The present invention also provides a kit for detecting or quantifying a pathogen by real-time nested PCR of the present invention. The kit comprises at least two oligonucleotide primers complementary to the nucleotide sequence of a pathogen to be amplified for obtaining a first amplified product, and at least two nested oligonucleotide primers complementary to the first amplified product for obtaining a second amplified product. The kit may further comprise a first compound that detects the first amplified product and/or a second compound that detects the second amplified product. The first and second compounds may be the same or different. The kit may further comprise a buffer, a positive control, a negative control, and/or a DNA polymerase. A positive control may be used to establish a standard curve and may be a known amount of an RNA or DNA pathogen, or a nucleotide sequence of the RNA or DNA pathogen to be tested. Alternatively, a positive control may include: (i) a nucleotide sequence other than the RNA or DNA pathogen to be tested, (ii) at least two oligonucleotide primers complementary to the nucleotide sequence for obtaining a first amplified product; (iii) and at least two nested oligonucleotide primers complementary to the first amplified product for obtaining a second amplified product. A negative control may be any nucleotide sequence or RNA/DNA pathogen unrelated to the pathogen to be tested. The negative control should not produce detectable levels of amplification product.

In an embodiment of the invention, the target nucleotide sequence in the first round real-time RT-PCR is the nucleotide sequence of SARS-CoV. The complete genome sequence of SARS-CoV has been determined (Marra M A et al., (2003) Science 300:1399-1404; Rota P A et al., (2003) Science 300:1394-1399). The methods of detecting or quantifying SARS-CoV in a sample are illustrated by the following Examples, which are not intended to be limiting in any way.

Before the present invention is further described, it is to be understood that this invention is not limited to particular embodiments described. It is also to be understood that the terminology used herein is for the purpose of describing particular embodiments only, and is not intended to be limiting, since the scope of the present invention will be limited only by the appended claims.

Where a range of values is provided, it is understood that intervening values are encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, certain methods and materials are now described. All publications mentioned herein are incorporated herein by reference to disclose and describe the methods and/or materials in connection with which the publications are cited.

It must be noted that as used herein and in the appended claims, the singular forms “a,” “or,” and “the” include plural referents unless the context clearly dictates otherwise.

Further, unless otherwise indicated, all numbers expressing quantities of ingredients, reaction conditions, % purity, primer lengths, and so forth, used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLE 1

### Reagents and Assays for Detecting or Quantifying SARS-CoV

Patient Specimens

Specimens of throat swabs from individuals suspected of having SARS were collected using the Venturi Transystem (Copan Diagnostics, Corona, USA) and subsequently used for viral RNA extraction. According to the World Health Organization's (WHO) definition (http://www.who.int/csr/sars/casedefinition/en/), a patient with a suspected case of SARS has high fever (temperature, >38° C.), cough or breathing difficulty, and a history of exposure to or contact with a person with suspected or probable SARS. In the following Examples, case patients were people who had symptoms similar to those as defined by the WHO, except that a history of their contact was not clear.

Viral Nucleic Acid Extraction

Viral RNA was extracted from 200 μl of viral transport medium (Venturi Transystem; Copan Diagnostics) with QIAamp viral RNA mini kit (Quiagen Inc., Valencia, USA) according to the instructions of the manufacturer and eluted in 50 μl of RNase free water.

Hybridization Probe-Based Single Round Real-Time RT-PCR

Single round RT-PCR using hybridization probes was performed using the RealArt HPA-coronavirus RT PCR Reagents kit (Artus Biotech, Germany) according to the manufacturer's instructions.

Positive Control

Control viral RNA was extracted from supernatant of a culture medium containing SARS-CoV. The titer of viral RNA was calibrated using RealArt HPA-coronavirus RT PCR Reagents kit as described above and a high titer RNA stock of 5.2×10⁵ copies/ml was generated. Samples containing different copy numbers of the viral RNA were created by adding 2 μl of a ten-fold serial dilution of the stock RNA.

Primers and RT-PCR Reagents

Two pairs of PCR primers, BNIoutS/BNIoutAS and BNIinS/BNIinAS as released in (http://www.who.int/csr/sars/primers/en/), were used for the first round RT-PCR and the subsequent nested PCR, respectively. Briefly, BNIoutS (5′ ATG AAT TAC CM GTC MT GGT TAC) (SEQ ID NO:1) and BNIoutAS (5′-CAT MC CAG TCG GTA CAG CTA C) (SEQ ID NO:2) were used to amplify a 195 bp region of the viral gene (orf1ab polyprotein), while the second round PCR amplification using BNIinS (5′-GM GCT ATT CGT CAC GTT CG) (SEQ ID NO:3) and BNIinAS (5′-CTG TAG AAA ATC CTA GCT GGA G) (SEQ ID NO:4) were used to amplify a 110-bp fragment from the first PCR product. The first round PCR amplification was performed in a one-step RT-PCR reaction using the LightCycler RNA master SYBR Green I kit (Roche Diagnostics GmbH, Germany). The reaction mixture contained 2 μl viral RNA, 2 mM Mn(OAC)I2 and 0.5 μM of BNIoutS/BNIoutAS primers. The real-time nested PCR reaction was then performed in a total reaction mixture of 20 μl using the LightCycler FastStart DNA Master SYBR Green I kit (Roche). The reaction mixture contained 2 mM of MgCl₂, 0.5 μM of BNIinS/BNIinAS primers and 1 μl of the first round amplicon as template.

LightCycler Settings

The first round real-time RT-PCR condition was optimized and the cDNA synthesis step was performed at 61° C. for 20 min and 95° C. for 30 seconds, followed by 25 cycles of 95° C. at 1 second, 55° C. for 10 seconds, 72° C. for 8 seconds. Real-time nested PCR was started at 95° C. for 10 minutes, followed by 25-35 cycles of 95° C. for 10 seconds, 56° C. for 5 seconds, and 72° C. for 5 seconds. After PCR amplification, a melting curve analysis was performed ranging from 65° C. to 95° C. with a temperature transition rate of 0.1° C./second.

Management of PCR Contamination

To reduce the risk of random contamination of nested PCR (Porter-Jordan K et al., (1990) J. Med. Virol. 30:85-91), sample preparation, reagent preparation, and PCR amplification were performed in different buildings or rooms with separated air-conditioning using different pipette systems. Furthermore, all used racks were treated by immersing in 1 N HCl and thoroughly drained. All samples and reagents were transferred via filter tips to protect from aerosol contamination of PCR samples and machines.

Confirmation of Amplification Products

The melting curve analysis allowed the determination of the melting point of the nested real-time PCR product and the presence of PCR amplified products. To confirm the size of the product, gel electrophoresis analysis in 2% agarose was performed. Furthermore, the resultant amplicon was subjected to sequence analysis using the ABI 3700 auto-sequencer (ABI) to confirm that it was part of the SARS-CoV sequence.

Data Analysis

Real-time nested PCR data were analyzed using the LightCycler Software Version 3.52. The baseline fluorescence derived from the fluorescence signal intensities of each cycle of the amplification was applied directly without adjustment. The threshold cycle (CT) was calculated and obtained by the “fit points” algorithm using a two point calculation. The noise band was moved to cross all sample curves in the lower log-linear part above the baseline noise, and the crossing point was then determined automatically for quantification. For quantification of the SARS-CoV RNA in the samples, a standard curve was generated using serial dilutions of a positive control (see “Positive control” above).

Seroconversion Analysis

Seroconversion refers to the development of antibodies against an antigen. Thus, detection of seroconversion is one method for confirming PCR positive results. Occurrence of seroconversion was determined by ELISA using serum samples obtained from patients during their convalescent phase of infection (>28 days after illness) (Peiris J S et al. (2003) Lancet 361:1767-1772). The SARS ELISA antigen was kindly provided by the US Centers for Disease Control and Prevention (Atlanta). The optimal dilution (1:1000) for the use of this antigen was determined by checkerboard titration against human serum samples obtained during the convalescent phase. The negative control antigen was prepared from uninfected Vero E6 cells (American Tissue Culture Collection) and was used to control for the specific reactivity of tested serum. The conjugates used were goat anti-human IgG, IgA, and IgM conjugated to fluorescein isothiocyanate (Jackson ImmunoResearch Labs) and horseradish peroxidase (BioRad) for the indirect fluorescence antibody test and ELISA, respectively.

### EXAMPLE 2

### Detection of One Copy of SARS-CoV by Real-Time Nested PCR

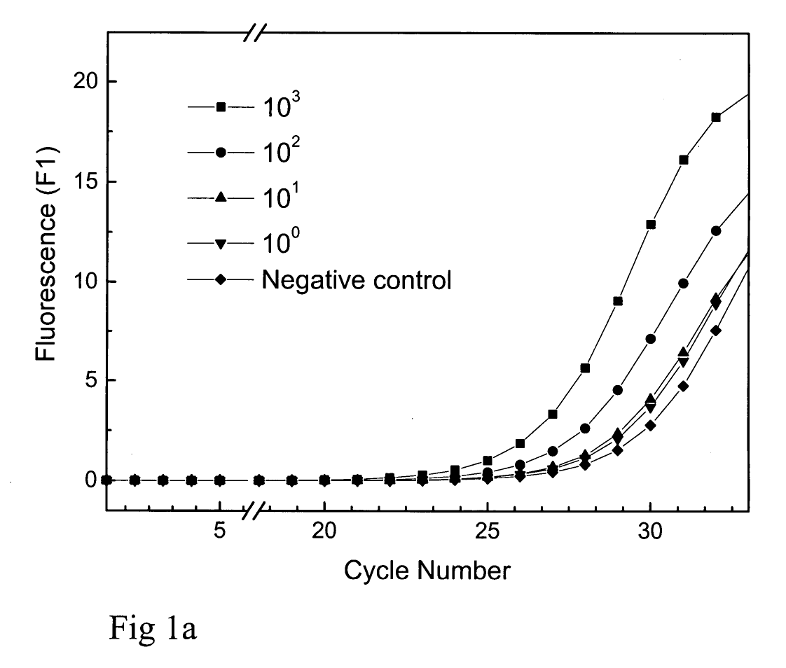

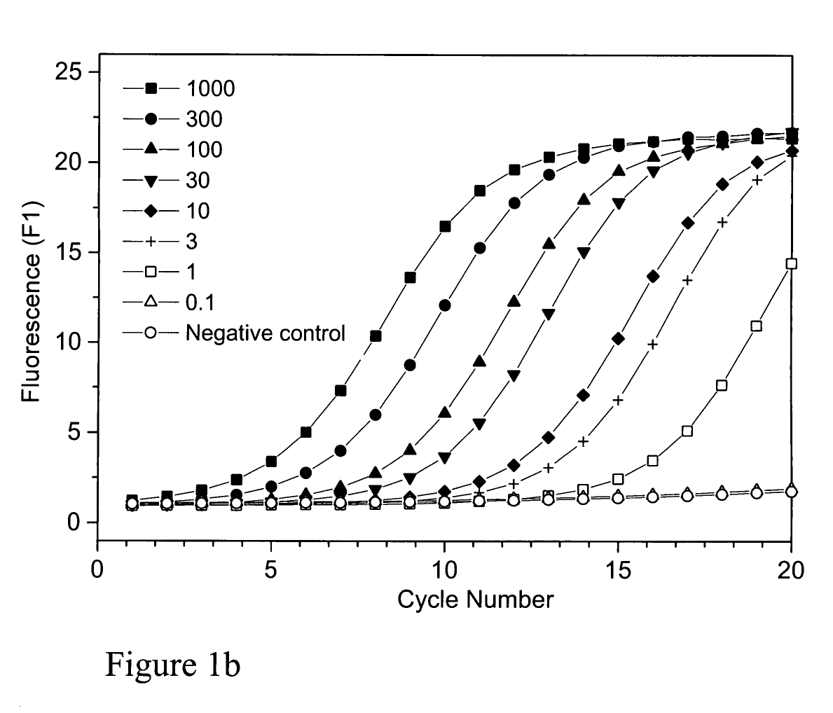

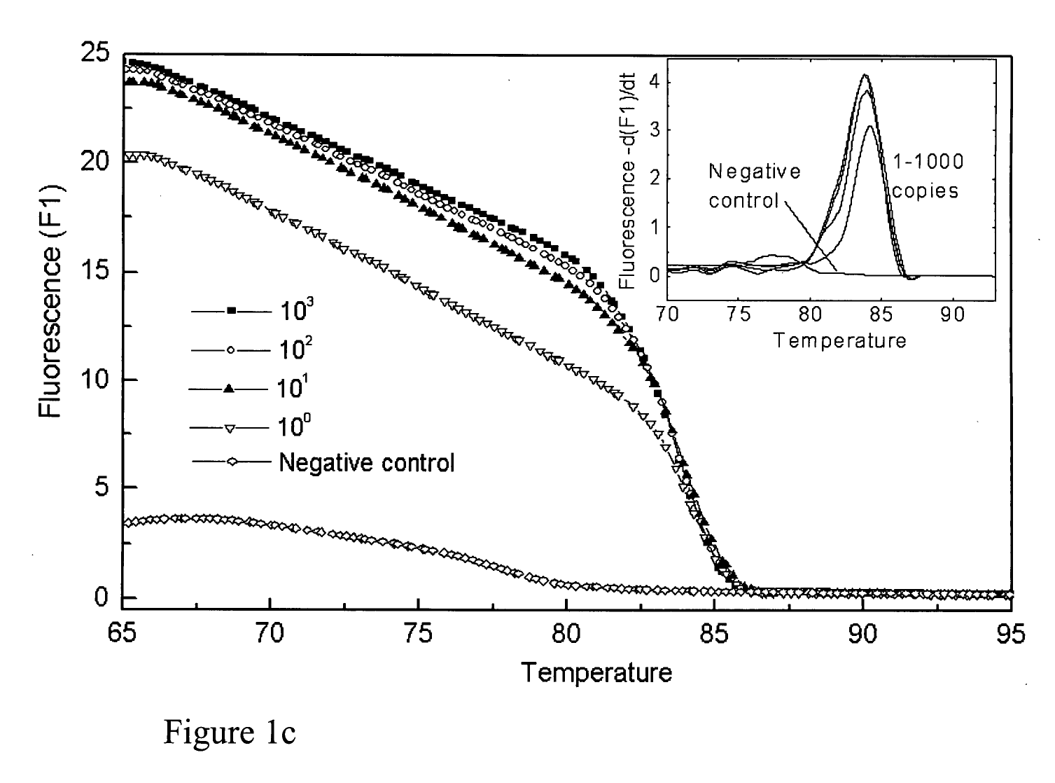

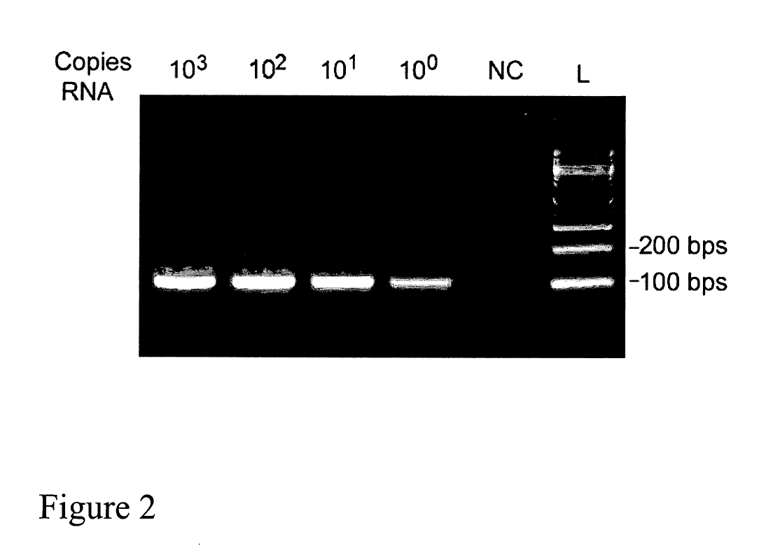

Using the LightCycler, the first round real-time PCR yielded a minor amplification signal (FIG. 1A, 10⁰ to 10³ copies RNA; predicted size of 190 bps), though the non-specific fluorescence signal background frequently occurred after 20 cycles of amplification (FIG. 1A, negative control). In contrast, the second run of nested real-time PCR efficiently amplified a signal of SARS-CoV DNA without any apparent background (FIG. 1B), which was comparable to the signal generated by the negative control samples (FIG. 1B, negative control). In addition, the melting curve analysis revealed a melting temperature (Tm) of 84.2° C. for the nested amplicon (FIG. 1C, 10⁰ to 10³ copies RNA) in contrast to no identifiable melting temperature in the negative control (FIG. 1C, negative control). The size (110 bps) of all nested PCR amplicons were confirmed by the agarose gel analysis (FIG. 2), and sequence analysis confirmed that they were SARS-CoV sequences.

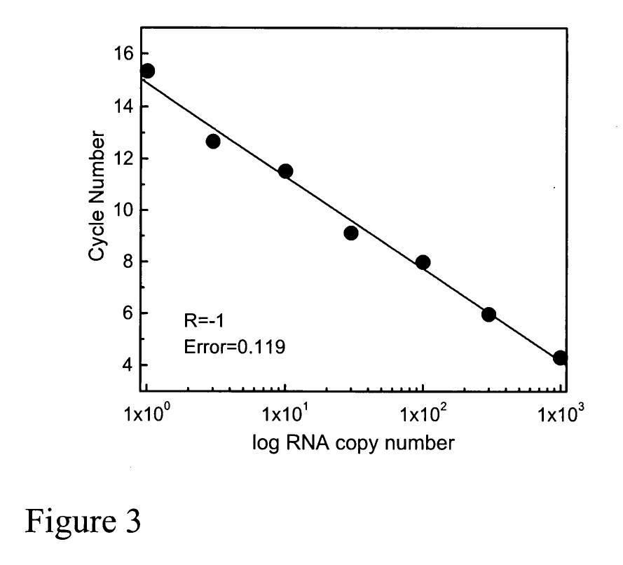

To determine the detection limit of the method of the invention, samples containing serially diluted control SARS-CoV RNA ranging from 10⁴ to 10⁰ copies/ml were subjected to the assay. After 25 cycles of real-time first-round amplification and 20 cycles of real-time nested PCR amplification, the assay could detect a single copy of extracted viral RNA which was clearly distinguishable from the negative control (FIG. 1B). The method of the invention exhibited superior sensitivity to other known methods for the detection of SARS-CoV at very low titer (see Example 3), which is characteristic of SARS-CoV at the early stages of infection. The optimal number of cycles in the real-time first-round RT-PCR was less than about 30 cycles in order to prevent non-linear amplification as reflected by saturation of the fluorescence signal (FIG. 1A). In most cases, 25 cycles produced adequate amplicon as the template for the subsequent real-time nested PCR (data not shown). A virtually perfect linear relationship was observed between the log copy number of input RNA and second-round cycle number when the log copy number of input RNA was within the range of 10³ to 10⁰ (FIG. 3). Thus, the method of the invention is a highly sensitive and specific method for detecting trace amounts of SARS-CoV and is also useful for quantifying the viral load.

In addition to the aforementioned primer sets, other PCR and nested PCR primer sets were used and showed comparable or even improved results, indicating other primer sets may be also suitable for use in this assay. For example, the following primer sets each comprising a pair of outer oligonucleotide primers and a pair of nested oligonucleotide primers may be used:

| | |
|---|---|
| Outer forward oligonucleotide: | |
| ggccgcaaattgcacaatttgctc | (SEQ ID NO:5) |
| Outer reverse oligonucleotide: | |
| ccatgtcagccgcaggaagaagag | (SEQ ID NO:6) |
| Nested forward oligonucleotide: | |
| tgcctctgcattctttggaatgtc | (SEQ ID NO:7) |
| Nested reverse oligonucleotide: | |
| tatgcgtcaatgtgcttgttcagc | (SEQ ID NO:8) |
| Outer forward oligonucleotide: | |
| catggcaaggaggaacttagattc | (SEQ ID NO:9) |
| Outer reverse oligonucleotide: | |
| cacggtggcagcattgttattagg | (SEQ ID NO:10) |
| Nested forward oligonucleotide: | |
| acaccaatagtggtccagatgacc | (SEQ ID NO:11) |
| Nested outer oligonucleotide: | |
| cgccgtagggaagtgaagcttctg | (SEQ ID NO:12) |

Both primer sets hybridize to and amplify the N region (the putative nucleocapsid protein) of SARS-CoV. The outer oligonucleotide primers in both primer sets produce amplicons of 300 bps and the nested oligonucleotide primers in both primer sets produce amplicons of 150 bps.

### EXAMPLE 3

### The Real-Time Nested PCR Method is More Sensitive Than Conventional Assays

The two-round real-time PCR method was compared with a conventional hybridization probe-based single-round RT-PCR method using RNA samples extracted from 46 individuals suspected of having SARS. Results are shown in Table 1. Conventional single-round PCR detected 15/46 positive cases while the two-round real-time PCR method of the invention detected 17/46 positive cases. Among these 17 positive cases, 15 were identical to those detected by the conventional single-round PCR. For the two additional cases detected by the two-round real-time PCR method of the invention, direct sequencing analysis confirmed that they were true positives (data not shown). The two additional positives were also tested for their seroconversions. See Table 1. Both of these positives had seroconverted, further confirming that these two samples were true positives (see Table 1, under “Real-time nested PCR” for “Seroconversion” of “<10 copies per test”). It is highly unlikely that these two additional positives were due to contamination because carry-over contamination was carefully avoided. Moreover, the tests were confirmed by three independent tests.

Interestingly, virus titers of the aforementioned two additional positive cases were quantified to contain less than 10 copies of viral genome in the sample. In the 15 positive cases commonly detected by both the conventional assay and the method of the invention, one contained less than 10 copies of viral genome, indicating that the method of the invention is superior at detecting low titers of SARS-CoV.

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

TABLE 1
Comparison of results of real-time nested PCR and
single-round RT-PCR test for clinical samples from 46 patients.

| | Positive result^{a} | | | |
|---|---|---|---|---|
| | ≧10 copies | <10 copies | Negative | |
| Test | per test | per test | result | Total |
| Real-time nested PCR | | | | |
| No. of results | 14 | 3 | 29 | 46 |
| Seroconversion^{b} | 5 | 2 | 0 | 7 |
| Single-round RT-PCR^{c} | | | | |
| No. of results | 14 | 1 | 31 | 46 |
| Seroconversion^{b} | 5 | 0 | 2 | 7 |
| Shared results | | | | |
| No. of results | 14 | 1 | 29 | 44 |
| Seroconversion^{b} | 5 | 0 | 0 | 5 |

NOTE.
Data are no. of RNA samples. RNA samples were extracted from 46 clinical throat swab specimens and analyzed by real-time nested PCR and single-round RT-PCR in parallel.
^{a}Results of 3 independent tests.
^{b}Data are no. of samples obtained from patients for whom seroconversion was also noted, as determined on the basis of ELISA results for available serum samples obtained during the convalescent phase of illness (i.e., >28 days after onset of illness).
^{c}Results obtained by hybridization probe-based detection using the RealArt HPA-coronavirus RT-PCR reagents kit (Artus).

1. A method of detecting an RNA pathogen in a sample, comprising: subjecting a sample suspected of containing the RNA pathogen to real-time nested PCR, wherein the real-time nested PCR comprises the steps of:
(a) subjecting the sample to real-time reverse transcriptase-polymerase chain reaction (RT-PCR) in the presence of at least two oligonucleotide primers complementary to a nucleotide sequence of the RNA pathogen and obtaining a first amplified product; wherein the first amplified product is a product of linear amplification;
(b) subjecting the first amplified product to real-time PCR in the presence of at least two nested oligonucleotide primers complementary to the nucleotide sequence of the first amplified product and obtaining a second amplified product;
(c) and detecting the second amplified product, wherein the second amplified product indicates the presence of RNA pathogen in the sample.

2. The method of claim 1, wherein the RNA pathogen is selected from togavirus, coronavirus, astrovirus, picornavirus, and retrovirus.

3. The method of claim 1, wherein the RNA pathogen is SARS-CoV.

4. The method of claim 1, wherein the RNA pathogen is human immunodeficiency virus.

5. A method of quantifying an RNA pathogen in a sample, comprising subjecting a sample suspected of containing the RNA pathogen to real-time nested PCR, wherein the real-time nested PCR comprises the steps of:
(a) subjecting the sample to real-time reverse transcriptase-polymerase chain reaction (RT-PCR) in the presence of at least two oligonucleotide primers complementary to a nucleotide sequence of the RNA pathogen and obtaining a first amplified product; wherein the first amplified product is a product of linear amplification;
(b) subjecting the first amplified product to real-time PCR in the presence of at least two nested oligonucleotide primers complementary to the nucleotide sequence of the first amplified product and obtaining a second amplified product;
(c) detecting the second amplified product; and
(d) quantifying the amount of the RNA pathogen contained in the sample.

6. The method of claim 1 or 5, wherein the first and second amplified products are detected by fluorescence.

7. The method of claim 1 or 5, wherein the first and second amplified products are detected by light scatter.

8. The method of claim 5, wherein the RNA pathogen is selected from togavirus, coronavirus, astrovirus, picornavirus, and retrovirus.

9. The method of claim 5, wherein the RNA pathogen is SARS-CoV.

10. The method of claim 5, wherein the RNA pathogen is human immunodeficiency virus.

11. The method of claims 3 or 9, wherein the at least two oligonucleotide primers in step (a) have the sequence SEQ ID NO:1 and SEQ ID NO:2.

12. The method of claims 3 or 9, wherein the at least two nested oligonucleotide primers in step (b) have the sequence SEQ ID NO:3 and SEQ ID NO:4.

13. The method of claim 1, wherein the method detects less than 10 copies of the RNA pathogen.

14. The method of claim 5, where the method quantifies less than 10 copies of the RNA pathogen.

15. A method of detecting a DNA pathogen in a sample, comprising: subjecting a sample suspected of containing the DNA pathogen to real-time nested PCR, wherein the real-time nested PCR comprises the steps of:
(a) subjecting the sample to real-time polymerase chain reaction (PCR) in the presence of at least two oligonucleotide primers complementary to a nucleotide sequence of the DNA pathogen and obtaining a first amplification product, wherein the first amplification product is a product of linear amplification;
(b) subjecting the first amplification product to real-time PCR in the presence of at least two nested oligonucleotide primers complementary to the nucleotide sequence of the first amplification product and obtaining a second amplification product;
(c) and detecting the second amplified product, wherein the second amplified product indicates the presence of DNA pathogen in the sample.

16. The method of claim 15, wherein the DNA pathogen is selected from parovirus, papovavirus, polyomavirus, adenovirus, herpes virus, and hepadnavirus.

17. The method of claim 15, wherein the DNA pathogen is a bacteria or yeast.

18. The method of claim 15, wherein the method detects less than 10 copies of the DNA pathogen.

19. A method of quantifying a DNA pathogen in a sample, comprising subjecting a sample suspected of containing the DNA pathogen to real-time nested PCR, wherein the real-time nested PCR comprises the steps of:
(a) subjecting the sample to real-time polymerase chain reaction (PCR) in the presence of at least two oligonucleotide primers complementary to a nucleotide sequence of the DNA pathogen and obtaining a first amplification product, wherein the first amplification product is a product of linear amplification;
(b) subjecting the first amplification product to real-time PCR in the presence of at least two nested oligonucleotide primers complementary to the nucleotide sequence of the first amplification product and obtaining a second amplification product;
(c) detecting the second amplified product; and
(d) quantifying the amount of the DNA pathogen contained in the sample.

20. The method of claim 15 or 19, wherein the first and second amplified products are detected by fluorescence.

21. The method of claim 15 or 19, wherein the first and second amplified products are detected by light scatter.

22. The method of claim 19, wherein the DNA pathogen is selected from parovirus, papovavirus, polyomavirus, adenovirus, herpes virus, and hepadnavirus.

23. The method of claim 19, wherein the DNA pathogen is a bacteria or yeast.

24. The method of claim 19, where the method quantifies less than 10 copies of the DNA pathogen.

25. A composition comprising:
(a) a first amplified product obtained from subjecting a sample containing an RNA pathogen to real-time reverse transcriptase-polymerase chain reaction (RT-PCR) in the presence of at least two oligonucleotide primers complementary to a nucleotide sequence of the RNA pathogen; wherein the first amplified product is a product of linear amplification;
(b) at least two nested oligonucleotide primers complementary to the nucleotide sequence of the first amplified product; and
(c) a compound that detects a second amplification product obtained from nested PCR amplification of the first amplified product using the nested oligonucleotide primers of (b).

26. The composition of claim 25, wherein the compound of (c) is a fluorogenic molecule that binds to double-stranded DNA.

27. The composition of claim 25, wherein the compound of (c) induces light scatter.

28. The composition of claim 25, wherein the RNA pathogen is selected from togavirus, coronavirus, astrovirus, picornavirus, and retrovirus.

29. The composition of claim 25, wherein the RNA pathogen is SARS-CoV.

30. The composition of claim 25, wherein the RNA pathogen is human immunodeficiency virus.

31. A composition comprising:
(a) a first amplified product obtained from subjecting a sample containing an DNA pathogen to real-time polymerase chain reaction (PCR) in the presence of at least two oligonucleotide primers complementary to a nucleotide sequence of the DNA pathogen; wherein the first amplified product is a product of linear amplification;
(b) at least two nested oligonucleotide primers complementary to the nucleotide sequence of the first amplified product; and
(c) a compound that detects a second amplification product obtained from nested PCR amplification of the first amplified product using the nested oligonucleotide primers of (b).

32. The composition of claim 31, wherein the compound of (c) is a fluorogenic molecule that binds to double-stranded DNA.

33. The composition of claim 31, wherein the compound of (c) induces light scatter.

34. The composition of claim 31, wherein the DNA pathogen is selected from parovirus, papovavirus, polyomavirus, adenovirus, herpes virus, and hepadnavirus.

35. The composition of claim 31, wherein the DNA pathogen is a bacteria or yeast.

36. A kit for detecting or quantifying a RNA pathogen in a sample by real-time nested PCR comprising:
(a) at least two oligonucleotide primers complementary to a nucleotide sequence of the RNA pathogen, wherein the at least two oligonucleotide primers are used to obtain a first amplified product in a real-time reverse transcriptase polymerase chain reaction (RT-PCR);
(b) at least two nested oligonucleotide primers complementary to the nucleotide sequence of the first amplified product, wherein the at least two nested oligonucletide primers are used to obtain a second amplified product in a real-time nested PCR reaction; and
(c) a first compound that detects the first amplification product and a second compound that detects the second amplification product.

37. The kit of claim 36, wherein the first and second compounds are the same.

38. The kit of claim 37, wherein the first and second compounds of (c) are fluorogenic molecules that bind to double-stranded DNA.

39. The kit of claim 37, wherein the first and second compounds of (c) induce light scatter.

40. The kit of claim 36, wherein the RNA pathogen is selected from togavirus, coronavirus, astrovirus, picornavirus, and retrovirus.

41. The kit of claim 36, wherein the RNA pathogen is SARS-CoV.

42. The kit of claim 36, wherein the RNA pathogen is human immunodeficiency virus.

43. A kit for detecting or quantifying a DNA pathogen in a sample by real-time nested PCR comprising:
(a) at least two oligonucleotide primers complementary to a nucleotide sequence of the DNA pathogen, wherein the at least two oligonucleotide primers are used to obtain a first amplified product in a real-time polymerase chain reaction (PCR);
(b) at least two nested oligonucleotide primers complementary to the nucleotide sequence of the first amplified product, wherein the at least two nested oligonucletide primers are used to obtain a second amplified product in a real-time nested PCR reaction; and
(c) a first compound that detects the first amplification product and a second compound that detects the second amplification product.

44. The kit of claim 43, wherein the first and second compounds are the same.

45. The kit of claim 44, wherein the first and second compounds of (c) are fluorogenic molecules that bind to double-stranded DNA.

46. The kit of claim 44, wherein the first and second compounds of (c) induce light scatter.

47. The kit of claim 43, wherein the DNA pathogen is selected from parovirus, papovavirus, polyomavirus, adenovirus, herpes virus, and hepadnavirus.

48. The kit of claim 43, wherein the DNA pathogen is a bacteria or yeast.
